(19) United States
(12) Patent Application Publication
(10) Pub. No.: US 2018/0146929 A1
(43) Pub. Date: May 31, 2018

(54) DEVICE FOR PREDICTING VENTRICULAR ARRHYTHMIA AND METHOD THEREFOR

(71) Applicant: UNIVERSITY OF ULSAN FOUNDATION FOR INDUSTRY COOPERATION, Ulsan (KR)

(72) Inventors: Se Gyeong JOO, Seoul (KR); Hyo Jeong LEE, Ulsan (KR)

(73) Assignee: UNIVERSITY OF ULSAN FOUNDATION FOR INDUSTRY COOPERATION, Ulsan (KR)

(21) Appl. No.: 15/578,219

(22) PCT Filed: Jun. 1, 2016
(86) PCT No.: PCT/KR2016/005782

§ 371 Date: Nov. 29, 2017

(30) Foreign Application Priority Data
Jun. 1, 2015 (KR) 10-2015-0077305

(51) Int. Cl. A61B 5/00; A61B 5/0205; A61B 5/0456; A61B 5/04

(52) U.S. Cl. CPC A61B 5/7275; A61B 5/0205; A61B 5/02405; A61B 5/04012; A61B 5/7264; A61B 5/0456

(57) ABSTRACT

A method for predicting ventricular arrhythmia includes a step of receiving at least one of an electrocardiogram signal and a respiration signal of a ventricular arrhythmia patient; a step of acquiring at least one of parameter values for heart rate variability and respiratory variability of the ventricular arrhythmia patient by analyzing at least one of the electrocardiogram signal and the respiration signal of the ventricular arrhythmia patient; a step of generating a ventricular arrhythmia estimation algorithm for predicting whether or not ventricular arrhythmia occurs by using the acquired parameter values; a step of predicting whether or not ventricular arrhythmia of a user occurs by applying at least one of the parameter values for the heart rate variability and the respiratory variability of the user to the ventricular arrhythmia estimation algorithm; and a step of outputting prediction results as to whether or not the ventricular arrhythmia occurs.

### TECHNICAL FIELD

### Background Art

### (a) Field of the Invention

The present invention relates to a device for predicting ventricular arrhythmia and a method therefor, and more specifically, to a device for predicting ventricular arrhythmia by using heart rate variability and respiratory variability.

### (B) DESCRIPTION OF THE RELATED ART

The heart consists of two atria of the left atrium and the right atrium and two ventricles of the left ventricle and the right ventricle, and contracts and relaxes by electrical stimulation of the heart muscle. At this time, a case where there is an electrical signal in the ventricular tissue other than a normal conduction is called ventricular arrhythmia.

If ventricular arrhythmia occurs, an ability of the heart to discharge blood is reduced, resulting in reduction of the amount of blood that is exhaled, and thereby, respiratory difficult, dizziness, and syncope can occur. In addition, if malignant arrhythmia such as ventricular contraction, ventricular tachycardia, ventricular fibrillation occurs, in a moment, a cardiac function is completely paralyzed, and thereby, a person can soon die due to a heart attack. Accordingly, if ventricular arrhythmia occurs, immediate emergency treatment should be provided, the cause should be accurately identified, and thereby, the disease should be cured.

However, ventricular arrhythmias often occur suddenly in a patient, and the patient often dies before being cured in a hospital, so it is difficult to receive emergency treatment unless ventricular arrhythmias is predicted early.

Recently, various studies including use of big data for predicting early the ventricular arrhythmia have been performed, but since it is applied to hospitalized patients and early prediction time is shorter than a few, it is a difficult to secure sufficient time to cope with occurrence of the ventricular arrhythmia.

A technology of a background of the present invention is disclosed in Korean Patent Publication No. 10-2012-0133793 (published on Dec. 12, 2012).

### DISCLOSURE

### Technical Problem

An object of the present invention is to provide a device and a method for predicting ventricular tachyarrhythmia by using heart rate variability and respiratory variability.

### Technical Solution

According to one embodiment of the present invention, a method for predicting ventricular arrhythmia includes a step of receiving at least one of an electrocardiogram signal and a respiration signal of a ventricular arrhythmia patient, a step of acquiring at least one of parameter values for heart rate variability and respiratory variability of the ventricular arrhythmia patient by analyzing at least one of the electrocardiogram signal and the respiration signal of the ventricular arrhythmia patient, a step of generating a ventricular arrhythmia estimation algorithm for predicting whether or not ventricular arrhythmia occurs by using the acquired parameter values, a step of predicting whether or not ventricular arrhythmia of a user occurs by applying at least one of the parameter values for the heart rate variability and the respiratory variability of the user to the ventricular arrhythmia estimation algorithm, and a step of outputting prediction results as to whether or not the ventricular arrhythmia occurs.

A parameter for the heart rate variability may include at least one of a mean normal-normal interval, an NN interval standard deviation (SDNN), a square root of mean squared differences of successive NN intervals (RMSSD), proportion of interval differences of successive NN intervals greater than 50 ms (pNN50), intensity of a signal in a very low frequency domain between 0 and 0.04 Hz (VLF), intensity of a signal in a low frequency domain between 0.04 and 0.15 Hz (LF), intensity of a signal in a high frequency domain between 0.15 and 0.40 Hz (HF), and a ratio between LF and HF (LF/HF), short-term heart rate variability (SD1), long-term heart rate variability (SD2), and a ratio between the short-term heart rate variability and the long-term heart rate variability (SD1/SD2), and a parameter for the respiratory variability may include at least one of an average of respiratory periods (RPdM), a standard deviation of respiratory periods (RPdSD), and a ratio between RPdSD and RPdM (RPdV).

The step of acquiring parameter information may include a step of detecting R peak from the electrocardiogram signal and generating RR interval data, a step of removing ectopic beat from the RR interval data, and a step of acquiring a result value for the parameter by using the RR interval data in which the ectopic beat is removed.

In the step of removing ectopic beat from the RR interval data, in a case where a size of the RR interval is larger than a threshold value, a corresponding RR interval section is removed.

In the step of generating a ventricular arrhythmia estimation algorithm, the ventricular arrhythmia estimation algorithm is generated by inputting at least one of parameter values for heart rate variability and respiratory variability of the ventricular arrhythmia patient into an artificial neural network, and the artificial neural network includes one input layer, a plurality of hidden layers, and one output layer, and at least one of a parameter value for the heart beat variability and a parameter value for the heart beat variability is input to the input layer.

A device for predicting ventricular arrhythmia according to another embodiment of the present invention, includes an input unit that receives at least one of an electrocardiogram signal and a respiration signal of a ventricular arrhythmia patient, an acquisition unit that acquires at least one of parameter values for heart rate variability and respiratory variability of the ventricular arrhythmia patient by analyzing at least one of the electrocardiogram signal and the respiration of the ventricular arrhythmia patient, a generation unit that generates a ventricular arrhythmia estimation algorithm for predicting whether or not ventricular arrhythmia occurs by using the acquired parameter values, a prediction unit that predicts whether or not ventricular arrhythmia of a user occurs by applying at least one of the parameter values for the heart rate variability and the respiratory variability of the user to the ventricular arrhythmia estimation algorithm, and an output unit that outputs prediction results as to whether or not the ventricular arrhythmia occurs.

### Advantageous Effects

As such, according to the present invention, a method for predicting ventricular arrhythmia predicts occurrence of the ventricular arrhythmia with high probability before the ventricular arrhythmia occurs. Particularly, the method can allow prediction one hour before occurrence of the ventricular arrhythmia, and thus, it is possible to for a patient to have sufficient time to cope with the occurrence of the ventricular arrhythmia.

In addition, not only a service can be provided in cooperation with a patient monitoring device provided in a hospital and but also a service can be provided in cooperation with a u-health device such as a portable electrocardiogram measuring device or a portable breath measuring device. Accordingly, a patient can quickly cope with occurrence of the ventricular arrhythmias even in daily life.

### DESCRIPTION OF THE DRAWINGS

FIG. 1 is a configuration diagram of a device for predicting ventricular arrhythmia according to an embodiment of the present invention.

FIG. 2 is a flowchart illustrating the method for predicting ventricular arrhythmia according to the embodiment of the present invention.

FIG. 3 is a flowchart for explaining step S220 of FIG. 2 in detail.

FIG. 4A is a graph illustrating RR interval data according to the embodiment of the present invention.

FIG. 4B is a graph in which ectopic beat is removed from the RR interval data according to the present invention.

FIG. 4C is a graph illustrating data obtained by performing detrending or the like of the data in which the ectopic beat is removed, according to the embodiment of the present invention.

FIG. 4D is a graph illustrating power spectral density according to the embodiment of the present invention.

FIG. 5 is a diagram illustrating a structure of a ventricular arrhythmia prediction algorithm with respect to heart rate variability according to the embodiment of the present invention.

FIG. 6 is a graph illustrating ventricular arrhythmia prediction results according to the embodiment of the present invention.

### MODE FOR INVENTION

Hereinafter, exemplary embodiments of the present invention will be described in detail with reference to the accompanying drawings such that those skilled in the art to which the present invention pertains can readily implement. However, the present invention can be embodied in many different forms and is limited to the embodiments set forth herein. In order to clearly illustrate the present invention, parts not related to the description are omitted, and like parts are denoted by like reference numerals or symbols throughout the specification.

Throughout the specification, when an element “includes” an element, it means that the element can further include other elements, without excluding other elements unless specifically stated otherwise.

Then, embodiments of the present invention will be described in detail with reference to the accompanying drawings such that those skilled in the art can easily implement the present invention.

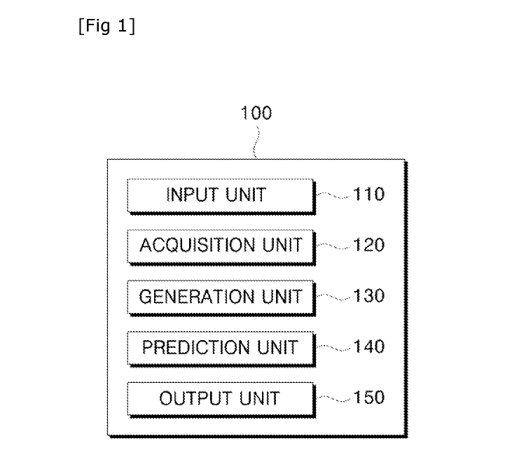

First, a configuration of a device 100 for predicting ventricular arrhythmia according to an embodiment of the present invention will be described with reference to FIG. 1. FIG. 1 is a configuration diagram of the device for predicting ventricular arrhythmia according to the embodiment of the present invention.

Referring to FIG. 1, the device for predicting ventricular arrhythmia 100 according to embodiment of the present invention includes an input unit 110, an acquisition unit 120, a generation unit 130, a prediction unit 140, and an output unit 150.

First, the input unit 110 receives a vital signal of a patient. At this time, the patient means a ventricular arrhythmia patient, and the vital signal of a patient includes at least one of an electrocardiogram signal (ECG signal) and a respiratory signal of the patient. In addition, in a case where the ventricular arrhythmia patient is normal, the vital signal of the patient includes a vital sign immediately before the ventricular arrhythmia occurs and a vital signal shortly after the ventricular arrhythmia occurs.

Next, the acquisition unit 120 analyzes the vital signal of the patient and acquires a parameter value for the vital variability. Here, the vital variability includes at least one of heart rate variability and respiratory variability.

Table 1 illustrates parameters for the heart rate variability and the respiratory rate variability.

TABLE 1

| Signal | Method | Parameter | Unit | Description |
|---|---|---|---|---|
| HRV | Time domain analysis | Mean NN | ms | Mean of NN interval |
| | | SDNN | ms | Standard deviation of NN intervals |
| | | RMSSD | ms | Square root of the mean squared |
| | | | | differences of successive NN intervals |
| | | pNN50 | % | Proportion of interval differences of |
| | | | | successive NN intervals greater than 50 ms |
| | Frequency domain analysis | VLF | ms² | Power in very low frequency range(0-0.04 |
| | | | | Hz) |
| | | LF | ms² | Power in low frequency range(0.04-0.15 |
| | | | | Hz) |
| | | HF | ms² | Power in high frequency range(0.15-0.4 |
| | | | | Hz) |
| | | LF/HF | | Ratio of LF over HF |
| | Nonlinear analysis | SD1 | ms | Standard deviation of the successive |
| | | | | intervals scaled by |
| | | | | $1 / \sqrt{2} \sqrt{\frac{1}{2} Var ( {RR}_{θ} - {RR}_{θ + 1}}$ |
| | | SD2 | ms | $\sqrt{2 {SDNN}^{2} - \frac{1}{2} {SD 1}^{2}}$ |
| | | SD1/SD2 | | Ratio of SD1 over SD2 |
| RRV | Time domain analysis | RPdM | ms | Respiration period mean(Mean of positive |
| | | | | peaks interval in respiration signal) |
| | | RPdSD | ms | Respiration period standard |
| | | | | deviation(Standard deviation of positive |
| | | | | peaks interval in respiration signal) |
| | | RPdV | | Respiration period variability |
| | | | | $\frac{RPdSD}{RPdM} \times 100$ |

Each parameter will be specifically described. First, the heart rate variability (HRV) parameter includes at least one of Mean NN, SDNN, RMSSD, pNN50, VLF, LF, HF, LF/HF, SD1, SD2, and SD1/SD2.

Here, parameter values of Mean NN, SDNN, RMSSD, and pNN50 are acquired through time domain analysis. Specifically, Mean NN means a mean normal-normal interval (NN interval), SDNN means a standard deviation of NN intervals, RMSSD means a square root of mean squared differences of successive NN intervals, and pNN50 means proportion of interval differences of successive NN intervals greater than 50 ms.

In addition, parameter values of VLF, LF, HF, and LF/HF are acquired through frequency domain analysis. Specifically, VLF (very low frequency) means intensity of a signal in a very low frequency domain between 0 and 0.04 Hz, LF (low frequency) means intensity of a signal in a low frequency domain between 0.04 and 0.15 Hz, HF (high frequency) means intensity of a signal in a high frequency domain between 0.15 and 0.40 Hz, and LF/HF means a ratio between LF and HF.

In addition, the parameter values of SD1, SD2, and SD1/SD2 are acquired through non-linear analysis. Specifically, SD1 (standard deviation 1) means short-term heart rate variability, SD2 (standard deviation 2) means long-term heart rate variability, and SD1/SD2 means a ratio between the short-term heart rate variability and the long-term heart rate variability.

Next, a respiratory rate variability (RRV) parameter includes at least one of RPdV, RPdSD, and RPdM. At this time, the parameter values of RPdV, RPdSD, and RPdM are acquired through time domain analysis. Specifically, respiration period mean (RPdM) means an average of respiratory periods, respiration period standard deviation (RPdSD) means a standard deviation of respiratory periods, and respiration period variability (RPdV) means a ratio between RPdSD and RPdM.

In addition, the generation unit 130 generates a ventricular arrhythmia prediction algorithm using the acquired parameter values of vital variability and an artificial neural network. Here, the ventricular arrhythmia prediction algorithm can be generated based on the artificial neural network. In addition, the ventricular arrhythmia prediction algorithm includes at least one of a ventricular arrhythmia prediction algorithm which uses a heart rate variability parameter, a ventricular arrhythmia prediction algorithm which uses a respiratory variability parameter, and a ventricular arrhythmia prediction algorithm which uses both the heart rate variability parameter and the respiratory variability parameter.

Next, the prediction unit 140 receives vital information of a user and applies the vital information of a user to the ventricular arrhythmia prediction algorithm to predict whether or not ventricular arrhythmia occurs on a user.

In addition, the output unit 150 outputs prediction results as to whether or not the ventricular arrhythmia occurs on the user. At this time, the prediction results can be displayed through a terminal of the user.

Meanwhile, according to the embodiment of the present invention, the prediction unit 140 and the output unit 150 can be realized as a device separately from the input unit 110, the acquisition unit 120, and the generation unit 130, or can be realized as a ventricular arrhythmia prediction server.

For example, in a case where the prediction unit 140 and the output unit 150 are realized as the ventricular arrhythmia prediction server, the ventricular arrhythmia prediction server receives at least one of the ventricular arrhythmia parameter value and the respiratory variability parameter value of a user from a user terminal and predicts whether or not the ventricular arrhythmia of a user occurs. In addition, the ventricular arrhythmia prediction server outputs the prediction results to the user terminal and provides the prediction results to the user.

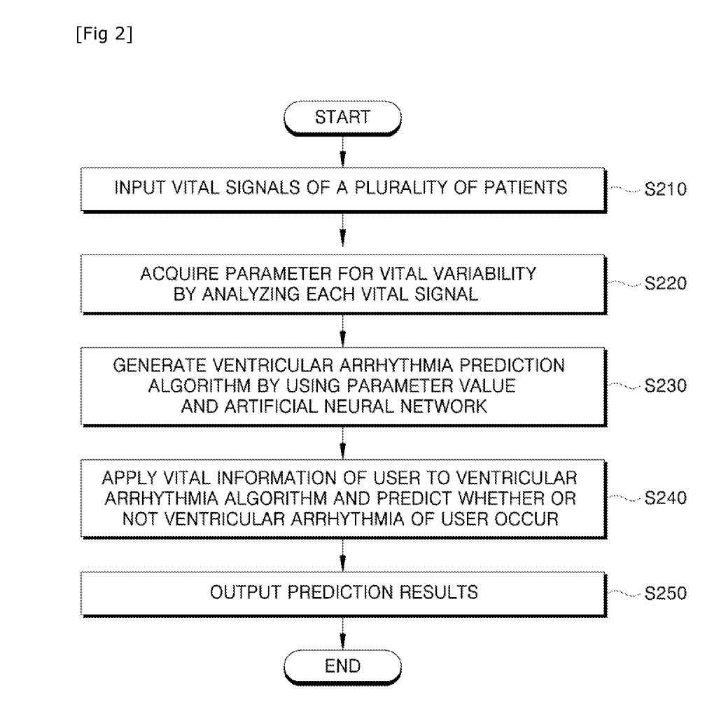

Hereinafter, a method for predicting ventricular arrhythmia using the device for predicting the ventricular arrhythmia according to the embodiment of the present invention will be described with reference to FIG. 2 through FIG. 5. FIG. 2 is a flowchart illustrating the method for predicting ventricular arrhythmia according to an embodiment of the present invention.

First, the input unit 110 receives vital signals of a plurality of ventricular arrhythmia patients (S210).

In addition, the acquisition unit 120 acquires parameter values for the vital variability by analyzing the input vital signals of the patients (S220).

According to the embodiment of the present invention, the acquisition unit 120 can detect an R peak from an electrocardiogram signal of the vital signals of the patients and generate RR interval data. In addition, the acquisition unit 120 can acquire a heart rate variability parameter value by using the generated RR interval data.

In addition, the acquisition unit 120 can generate respiratory peak interval data by detecting a respiratory peak from the respiration signal among the vital signals of the patients. The acquisition unit 120 can acquire the respiration variability parameter value by using the generated respiration peak interval data.

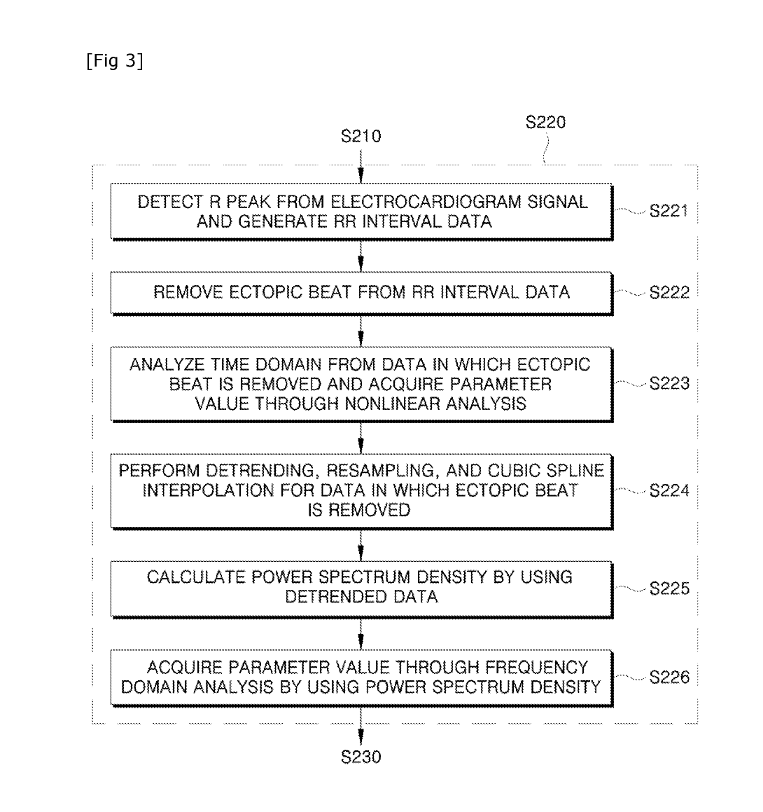

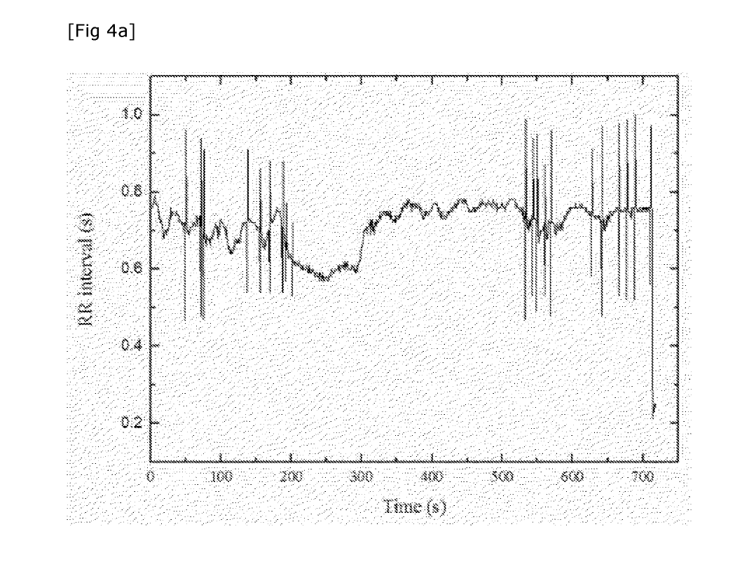

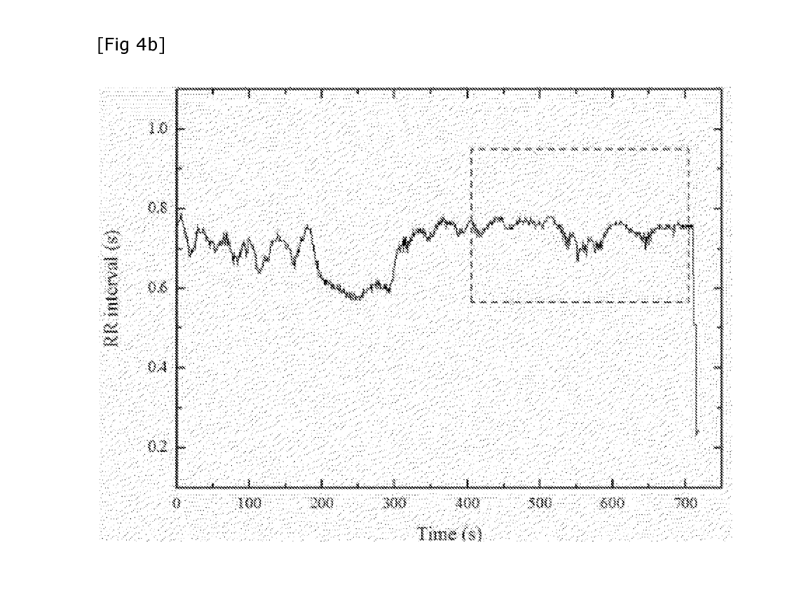

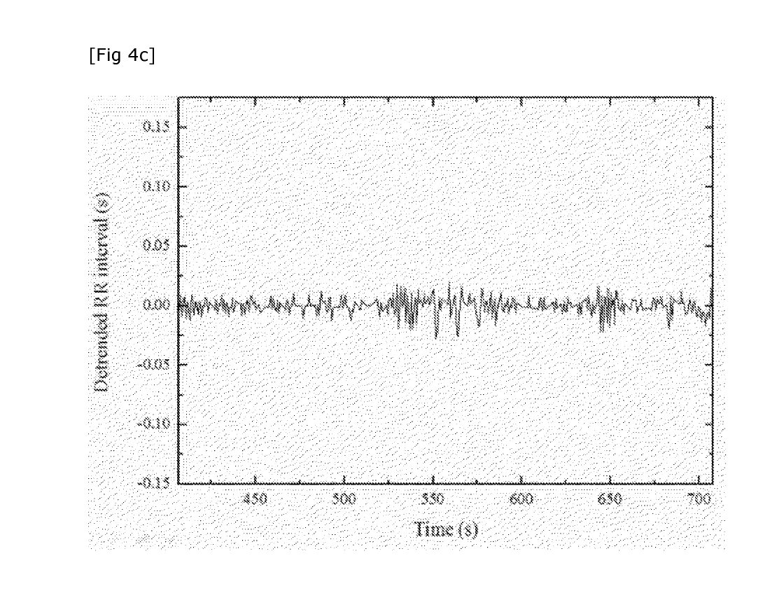

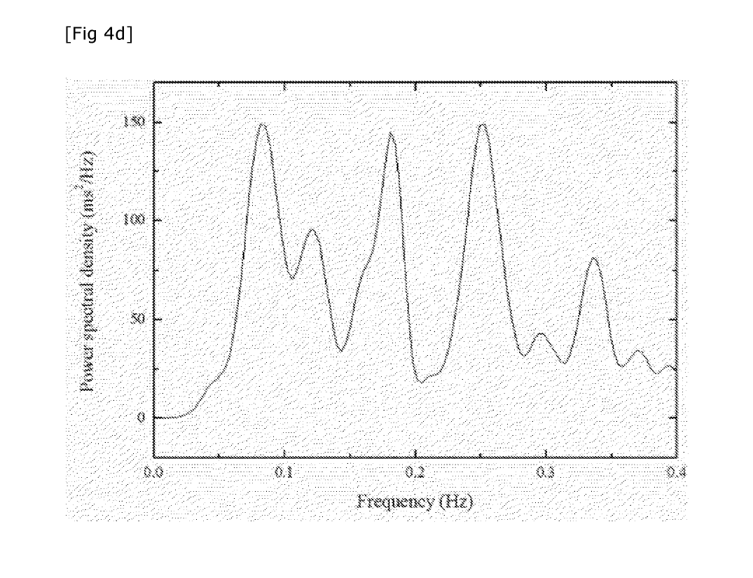

Then, a process of acquiring the parameter value for the heart rate variability which is step S220 will be described in detail with reference to FIGS. 3 to 4D. FIG. 3 is a flowchart illustrating step S220 of FIG. 2 in detail.

FIG. 4A is a graph illustrating the RR interval data according to the embodiment of the present invention, FIG. 4B is a graph in which ectopic beat is removed from the RR interval data according to the embodiment of the present invention, FIG. 4C is a graph illustrating data obtained by performing detrending or the like of the data in which the ectopic beat is removed, according to the present invention, and FIG. 4D is a graph illustrating power spectral density according to the embodiment of the present invention.

First, the acquisition unit 120 detects the R peak from the received electrocardiogram signal and generates the RR interval data (S221). Here, the RR interval means an interval between R-peaks of heart beat, which is also called an NN interval. Referring to FIG. 4A, the generated RR interval data can be represented by data having time as the x axis and having an RR interval as the y axis.

After the RR interval data is generated, the acquisition unit 120 removes the ectopic beat from the RR interval data (S222). The ectopic beat refers to a heart beat that irregularly appears once after a normal heart beat. As illustrated in FIG. 4A, a point where the RR interval appears irregularly and largely is a point where the ectopic heart beat appears.

The ectopic beat is removed by a method of removing the corresponding RR interval in a case where a size of the RR interval is larger than a threshold value. For example, assuming that the threshold value is 0.1, in a case where the RR interval is 0.2, the corresponding interval is removed, and in a case where the RR interval is 0.05, the corresponding interval is not removed. The acquisition unit 120 can acquire data having a form illustrated in FIG. 4B by removing an ectopic beat section from the RR interval data as illustrated in FIG. 4A.

Then, the acquisition unit 120 acquires parameter values for Mean NN, SDNN, RMSSD, and pNN50 from the RR interval data from which the ectopic beat is removed through time domain analysis, and acquired parameter values for SD1, SD2, and SD1/SD2 through non-linear analysis (S223).

Next, the acquisition unit 120 generates data for analysis data for frequency domain analysis by detrending, resampling, cubic spline interpolating, and power spectral density calculating the data in which the ectopic beat is removed (S224, S225).

Specifically, the acquisition unit 120 performs detrending of the data in which the ectopic beat is removed, by using a time-varying finite impulse response high-pass filter. At this time, the detrending means a data operation of removing a long-term trend of the data in which the ectopic beat is removed and of emphasizing a short-term change.

In addition, the acquisition unit 120 resamples the detrended data to 7 Hz and performs a cubic spline interpolation to generate the data for frequency domain analysis. Here, the cubic spline interpolation means an interpolation method of creating a cubic polynomial over all given points and of connecting two points with each other using a smooth curve. Data generated through the above-described process can be represented as a graph having a form illustrated in FIG. 4C.

In addition, after the detrending, the resampling, and the cubic spline interpolation are completed, the acquisition unit 120 calculates power spectral density (PSD), which can be represented as a graph having a form illustrated in FIG. 4B.

After the power spectral density is calculated in step S225, the acquisition unit 120 acquires parameter values for VLF, LF, and HF through the frequency domain analysis from the power spectral density (S226).

Table 2 illustrates parameter values for the vital variability acquired by the acquisition unit 120 through analysis of the vital signals.

TABLE 2

| | Control | | |
|---|---|---|---|
| | dataset (n = 110) | VTAs dataset (n = 110) | |
| Parameters | Mean ± SD | Mean ± SD | p-Value |
| Mean NN (ms) | 0.695 ± 0.162 | 0.701 ± 0.175 | 0.316 |
| SDSS (ms) | 0.056 ± 0.041 | 0.061 ± 0.04 | 0.068 |
| RMSSD (ms) | 0.061 ± 0.052 | 0.066 ± 0.049 | 0.158 |
| pNN50 (%) | 0.182 ± 0.2 | 0.189 ± 0.186 | 0.299 |
| VLF (ms²) | 3.09E−05 ± 5.39E−05 | 3.66E−05 ± 7.22E−05 | 0.196 |
| LF (ms²) | 6.2E−04 ± 1.08E−03 | 6.72E−04 ± 1.14E−03 | 0.312 |
| HF (ms²) | 1.27E−03 ± 1.80E−03 | 1.35E−03 ± 1.76E−03 | 0.314 |
| LF/HF | 0.523 ± 0.637 | 0.554 ± 0.543 | 0.297 |
| SD1 (ms) | 0.036 ± 0.029 | 0.039 ± 0.028 | 0.127 |
| SD2 (ms) | 0.075 ± 0.055 | 0.082 ± 0.053 | 0.066 |
| SD1/SD2 | 0.455 ± 0.171 | 0.477 ± 0.166 | 0.052 |
| RPdM (ms) | 2.79 ± 0.802 | 2.85 ± 0.928 | 0.072 |
| RPdSD (ms) | 0.879 ± 0.768 | 0.892 ± 0.789 | 0.444 |
| RPdV | 34.1 ± 6.79 | 28.2 ± 2.58 | <0.002 |

Here, Mean means an average value, SD means a standard deviation, p-value means significance probability, and the significance probability (p-value) means probability that extreme results will be actually observed rather than results obtained when null hypothesis is true.

As such, after acquiring the parameter values for a vital variability through step S220, the generation unit 130 generates the ventricular arrhythmia prediction algorithm using the parameter value for the vital variability and an artificial neural network (S230).

Here, the artificial neural network (ANN) is a statistical learning algorithm inspired by a biological neural network (animal's central nervous system, particularly the brain), and indicates an overall model having problem solving ability by changing a binding strength of a synapse using artificial neurons (nodes), and, in the embodiment of the present invention, the ventricular arrhythmia prediction algorithm is generated based on the artificial neural network. The device 100 for predicting ventricular arrhythmia according to the embodiment of the present invention can use a machine learning algorithm such as a support vector machine (SVM) as well as the artificial neural network.

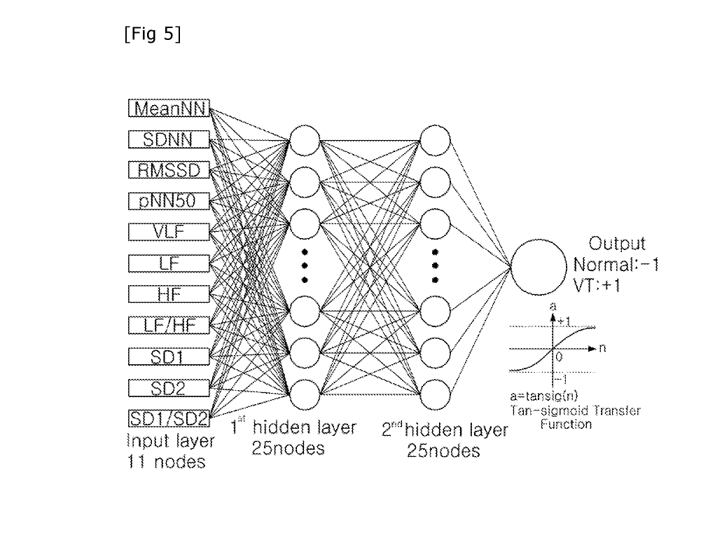

Then, a process of generating the ventricular arrhythmia prediction algorithm using parameters for the heart beat variability using the artificial neural network which is an embodiment of the present invention, will be described with reference to FIG. 5. FIG. 5 is a diagram illustrating a structure of the ventricular arrhythmia prediction algorithm for the heart rate variability according to an embodiment of the present invention.

As illustrated in FIG. 5, a node of a square and a node of a circle which are marked with parameters indicate artificial neurons. In addition, a connection line indicates an output from one neuron and an input to another neuron.

Specifically, the artificial neural network can be configured by an input layer including 11 nodes, a first hidden layer including 25 nodes, a second hidden layer including 25 nodes, and an output node. The artificial neural network can generate a ventricular arrhythmia prediction algorithm by learning to have a value of −1 when being normal and a value of +1 when being predicted to be ventricular arrhythmia for each parameter information.

At this time, a back propagation learning rule is used for the learning, and the backpropagation learning rule is a learning method of adjusting a weight so that a desired output value is activated as an input is given. In the embodiment of the present invention, the weight is adjusted such that the learning ends when a mean square error is less than 10⁻⁵.

Meanwhile, the ventricular arrhythmia prediction algorithm which uses parameters for the respiratory variability and the heart rate variability, and the ventricular arrhythmia prediction algorithm which uses parameters for the respiratory variability can also be generated by the same method as the method of generating the ventricular arrhythmia prediction algorithm which uses parameters for the heart rate variability.

After the ventricular arrhythmia prediction algorithm is generated in step S230, the prediction unit 140 receives vital information of a user and applies the vital information to an algorithm for ventricular arrhythmia prediction to predict whether or not the ventricular arrhythmia of the user occurs (S240), and outputs prediction results (S250). At this time, the vital information of the user can be acquired through a user terminal.

Meanwhile, steps S210 to S250 can be implemented by a computer-readable recording medium in which a program for performing the ventricular arrhythmia prediction method is recorded. In addition, steps S240 and S250 can be implemented by the computer-readable recording medium in which the program for performing the ventricular arrhythmia prediction method is recorded. The ventricular arrhythmia prediction algorithm generated in steps S210 to S230 can also be executed by a computer readable recording medium in which a program.

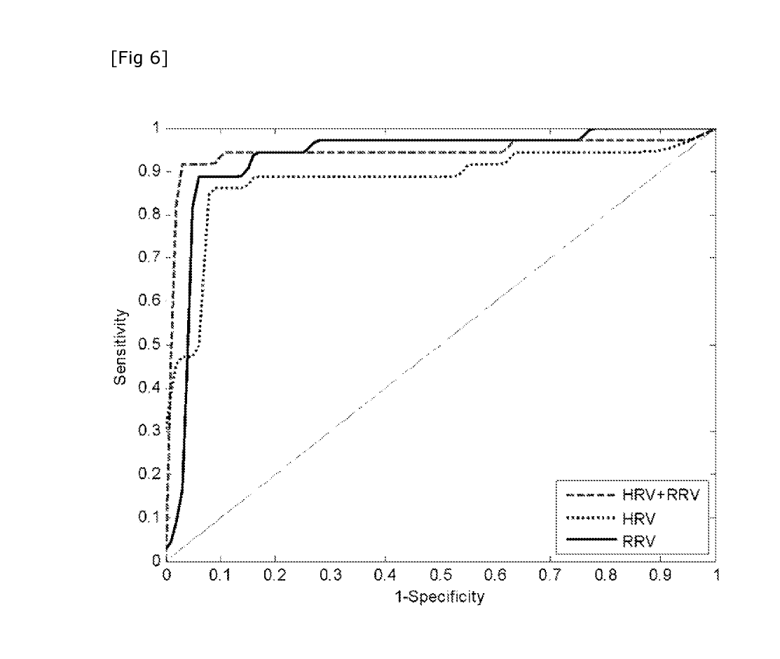

Hereinafter, the ventricular arrhythmia prediction results of a user according to the embodiment of the present invention will be described with reference to FIG. 6. FIG. 6 is a graph illustrating the ventricular arrhythmia prediction results according to the embodiment of the present invention.

First, Table 3 below illustrates results obtained by determining whether or not ventricular arrhythmia of a user occurs by using the method for predicting the ventricular arrhythmia according to the embodiment of the present invention.

TABLE 3

| ANN using | Input | Sensitivity (%) | Specificity (%) | Accuracy (%) | PPV (%) | NPV (%) | AUC |
|---|---|---|---|---|---|---|---|
| HRV | 11 | 86.1 (31/36) | 86.1 (31/36) | 86.1 (62/72) | 86.1 (31/36) | 86.1 (31/36) | 0.882 |
| parameters | | | | | | | |
| RRV | 3 | 91.7 (33/36) | 97.2 (35/36) | 94.4 (68/72) | 97.1 (33/34) | 92.1 (35/38) | 0.938 |
| parameters | | | | | | | |
| HRV + RRV | 14 | 91.7 (33/36) | 97.2 (35/36) | 94.4 (68/72) | 97.1 (33/34) | 92.1 (35/38) | 0.940 |
| parameters | | | | | | | |

As illustrated in Table 3, in a case of the ventricular arrhythmia prediction algorithm which uses the heart rate variability parameter, the ventricular arrhythmia prediction results indicate that accuracy is 86.1%, specificity is 86.1%, sensitivity is 86.1%, PPV (positive predictive value) probability is 86.1%, NPV (negative predictive value) probability is 86.1%, and AUC (area under the roccurve) is 0.882, and in a case of the ventricular arrhythmia prediction algorithm which uses the respiratory variability parameter, the ventricular arrhythmia prediction results indicate that accuracy is 94.4%, specificity is 97.2%, sensitivity is 91.7%, PPV (positive predictive value) probability is 86.1%, NPV (negative predictive value) probability is 86.1%, and AUC (area under the roccurve) is 0.938.

In addition, in a case of the algorithm which uses both the heart rate variability parameter and the respiratory variability parameter, the ventricular arrhythmia predictive results indicate that accuracy is 94.4%, specificity is 97.2%, sensitivity is 91.7%, PPV (positive predictive value) probability is 86.1%, NPV (negative predictive value) probability is 86.1%, and AUC (area under the roccurve) is 0.940.

Referring to FIG. 6, as a result of comparison of the AUC (area under the roccurve) illustrated in Table 3, in a case of the algorithm which uses both the heart rate variability parameter and the respiratory variability parameter has a better prediction performance than the algorithm which uses either the heart rate variability parameter or the respiratory variability parameter.

As such, according to the embodiment of the present invention, the method for predicting the ventricular arrhythmia predicts occurrence of the ventricular arrhythmia with high probability before the ventricular arrhythmia occurs. Particularly, the prediction can be made one hour before occurrence of the ventricular arrhythmia, so that a patient can secure sufficient time to cope with occurrence of the ventricular arrhythmia.

In addition, not only a service can be provided in cooperation with a patient monitoring device provided in a hospital, but also a service can be provided in cooperation with a u-health device such as a portable electrocardiogram measuring device or a portable breath measuring device. Accordingly, a patient can quickly cope with occurrence of the ventricular arrhythmias even in daily life.

While the present invention is described with reference to exemplary embodiments illustrated in drawings, those are merely examples, and it will be understood by the skilled in the art that various modifications and equivalent embodiments can be made from those. Accordingly, the true scope of the present invention should be determined by technical ideas of the appended claims.

1. A method for predicting ventricular arrhythmia which uses a device for predicting ventricular arrhythmia, the method comprising:
a step of receiving at least one of an electrocardiogram signal and a respiration signal of a ventricular arrhythmia patient;
a step of acquiring at least one of parameter values for heart rate variability and respiratory variability of the ventricular arrhythmia patient by analyzing at least one of the electrocardiogram signal and the respiration signal of the ventricular arrhythmia patient;
a step of generating a ventricular arrhythmia estimation algorithm for predicting whether or not ventricular arrhythmia occurs by using the acquired parameter values;
a step of predicting whether or not ventricular arrhythmia of a user occurs by applying at least one of the parameter values for the heart rate variability and the respiratory variability of the user to the ventricular arrhythmia estimation algorithm; and
a step of outputting prediction results as to whether or not the ventricular arrhythmia occurs.

2. The method for predicting ventricular arrhythmia of claim 1,
wherein a parameter for the heart rate variability includes at least one of a mean normal-normal interval, an NN interval standard deviation (SDNN), a square root of mean squared differences of successive NN intervals (RMSSD), proportion of interval differences of successive NN intervals greater than 50 ms (pNN50), intensity of a signal in a very low frequency domain between 0 and 0.04 Hz (VLF), intensity of a signal in a low frequency domain between 0.04 and 0.15 Hz (LF), intensity of a signal in a high frequency domain between 0.15 and 0.40 Hz (HF), and a ratio between LF and HF (LF/HF), short-term heart rate variability (SD1), long-term heart rate variability (SD2), and a ratio between the short-term heart rate variability and the long-term heart rate variability (SD1/SD2), and
wherein a parameter for the respiratory variability includes at least one of an average of respiratory periods (RPdM), a standard deviation of respiratory periods (RPdSD), and a ratio between RPdSD and RPdM (RPdV).

3. The method for predicting ventricular arrhythmia of claim 2, wherein the step of acquiring parameter information includes,
a step of detecting R peak from the electrocardiogram signal and generating RR interval data;
a step of removing ectopic beat from the RR interval data; and
a step of acquiring a result value for the parameter by using the RR interval data in which the ectopic beat is removed.

4. The method for predicting ventricular arrhythmia of claim 3, wherein, in the step of removing ectopic beat from the RR interval data, in a case where a size of the RR interval is larger than a threshold value, a corresponding RR interval section is removed.

5. The method for predicting ventricular arrhythmia of claim 1,
wherein, in the step of generating a ventricular arrhythmia estimation algorithm, the ventricular arrhythmia estimation algorithm is generated by inputting at least one of parameter values for heart rate variability and respiratory variability of the ventricular arrhythmia patient into an artificial neural network, and
wherein the artificial neural network includes one input layer, a plurality of hidden layers, and one output layer, and at least one of a parameter value for the heart beat variability and a parameter value for the heart beat variability is input to the input layer.

6. A device for predicting ventricular arrhythmia comprising:
an input unit that receives at least one of an electrocardiogram signal and a respiration signal of a ventricular arrhythmia patient;
an acquisition unit that acquires at least one of parameter values for heart rate variability and respiratory variability of the ventricular arrhythmia patient by analyzing at least one of the electrocardiogram signal and the respiration of the ventricular arrhythmia patient;
a generation unit that generates a ventricular arrhythmia estimation algorithm for predicting whether or not ventricular arrhythmia occurs by using the acquired parameter values;
a prediction unit that predicts whether or not ventricular arrhythmia of a user occurs by applying at least one of the parameter values for the heart rate variability and the respiratory variability of the user to the ventricular arrhythmia estimation algorithm; and
an output unit that outputs prediction results as to whether or not the ventricular arrhythmia occurs.

7. The device for predicting ventricular arrhythmia of claim 6, wherein a parameter for the heart rate variability includes at least one of a mean normal-normal interval, an NN interval standard deviation (SDNN), a square root of mean squared differences of successive NN intervals (RMSSD), proportion of interval differences of successive NN intervals greater than 50 ms (pNN50), intensity of a signal in a very low frequency domain between 0 and 0.04 Hz (VLF), intensity of a signal in a low frequency domain between 0.04 and 0.15 Hz (LF), intensity of a signal in a high frequency domain between 0.15 and 0.40 Hz (HF), and a ratio between LF and HF (LF/HF), short-term heart rate variability (SD1), long-term heart rate variability (SD2), and a ratio between the short-term heart rate variability and the long-term heart rate variability (SD1/SD2).

8. The device for predicting ventricular arrhythmia of claim 7, wherein a parameter for the respiratory variability includes at least one of an average of respiratory periods (RPdM), a standard deviation of respiratory periods (RPdSD), and a ratio between RPdSD and RPdM (RPdV).

9. The device for predicting ventricular arrhythmia of claim 8, wherein the acquisition unit detects R peak from the electrocardiogram signal and generates RR interval data, removes ectopic beat from the RR interval data, and acquires a result value for the parameter by using the RR interval data in which the ectopic beat is removed.

10. The device for predicting ventricular arrhythmia of claim 9, wherein, in a case where a size of the RR interval is larger than a threshold value, the acquisition unit removes ectopic beat from the RR interval data by removing a corresponding RR interval section.

11. The device for predicting ventricular arrhythmia of claim 6, wherein the generation unit generates the ventricular arrhythmia estimation algorithm by inputting at least one of parameter values for heart rate variability and respiratory variability of the ventricular arrhythmia patient into an artificial neural network, and
wherein the artificial neural network includes one input layer, a plurality of hidden layers, and one output layer, and at least one of a parameter value for the heart beat variability and a parameter value for the heart beat variability is input to the input layer.

12. A device for predicting ventricular arrhythmia comprising:
a prediction unit that applies at least one of parameter values for heart rate variability and respiratory variability of a user to a ventricular arrhythmia estimation algorithm and predicts whether or not ventricular arrhythmia of the user occurs; and
an output unit that outputs prediction results as to whether or not the ventricular arrhythmia occurs,
wherein the ventricular arrhythmia estimation algorithm analyzes at least one of an electrocardiogram signal and respiration of a ventricular arrhythmia patient, acquire at least one of parameter values for heart rate variability and respiratory variability of the ventricular arrhythmia patient, and is generated by using the parameter value which is acquired.

13. A service server for predicting ventricular arrhythmia,
wherein the service server applies at least one of parameter values for heart rate variability and respiratory variability of a user to a ventricular arrhythmia estimation algorithm, predicts whether or not ventricular arrhythmia of the user occurs, and outputs prediction results as to whether or not the ventricular arrhythmia occurs,
wherein the ventricular arrhythmia estimation algorithm is generated by using at least one of parameter values for heart rate variability and respiratory variability of the ventricular arrhythmia patient which are acquired by analyzing at least one of an electrocardiogram signal and respiration of a ventricular arrhythmia patient.

14. A method for predicting ventricular arrhythmia, comprising:
a step of applying at least one of parameter values for heart rate variability and respiratory variability of a user to a ventricular arrhythmia estimation algorithm, and predicting whether or not ventricular arrhythmia of the user occurs; and
a step of outputting prediction results as to whether or not the ventricular arrhythmia occurs,
wherein the ventricular arrhythmia estimation algorithm is generated by using at least one of parameter values for heart rate variability and respiratory variability of the ventricular arrhythmia patient which are acquired by analyzing at least one of an electrocardiogram signal and respiration of a ventricular arrhythmia patient.

15. A computer-readable recording medium in which a program for performing the method for predicting ventricular arrhythmia of claim 1 is recorded.

16. A computer-readable recording medium in which a program for performing the method for predicting ventricular arrhythmia of claim 14 is recorded.
